(19) United States
(12) Patent Application Publication
(10) Pub. No.: US 2025/0171393 A1
(43) Pub. Date: May 29, 2025

(54) METHOD FOR PRODUCING FORMATE AND METHOD FOR PRODUCING FORMIC ACID

(71) Applicant: NITTO DENKO CORPORATION, Ibaraki-shi, Osaka (JP)

(72) Inventors: Makoto HIRANO, Ibaraki-shi, Osaka (JP); Soichiro KOGA, Ibaraki-shi, Osaka (JP); Motosuke KATAYAMA, Ibaraki-shi, Osaka (JP); Jumpei KUNO, Ibaraki-shi, Osaka (JP); Masaya SERIU, Ibaraki-shi, Osaka (JP)

(21) Appl. No.: 18/840,951

(22) PCT Filed: Feb. 22, 2023
(86) PCT No.: PCT/JP2023/006380

§ 371 Date: Aug. 23, 2024

(30) Foreign Application Priority Data
Feb. 25, 2022 (JP) 2022-027472
Jun. 21, 2022 (JP) 2022-099864

(51) Int. Cl. C07C 51/02; B01J 31/20; B01J 31/22; B01J 31/24; B01J 37/00; B01J 37/03; B01J 37/04; B01J 37/08

(52) U.S. Cl. CPC C07C 51/02; B01J 31/20; B01J 31/2295; B01J 31/24; B01J 37/009; B01J 37/031; B01J 37/04; B01J 37/08; B01J 2531/004; B01J 2531/821

(57) ABSTRACT

The present invention provides a method for producing a formate, which is suitable for improving a TON of a metal catalyst. The production method of the present invention is a method for producing a formate through reaction between hydrogen, and a compound C including at least one selected from the group consisting of carbon dioxide, hydrogencarbonate, and carbonate in the presence of a solvent by using a metal catalyst. The solvent includes an organic solvent and an aqueous solvent. A ligand is added to the solvent as necessary. The reaction between hydrogen and the compound C is performed in a two-phase system in which the organic solvent and the aqueous solvent are separate under a condition that a value of y calculated by Equation (I) indicated below is more than 5.2.

$$y = 0.00398 {x}_{1} + ( - 0.0598 ) {x}_{2} + ( - 15.1 ) {x}_{3} + 6 \text{.66} {x}_{4} + ( - 0.00340 ) {x}_{5} + ( - 157 \; 99 ) {x}_{6} + 3.43 \times 1 {0}^{- 8} {x}_{7} + 4.97 \qquad ( I )$$

### TECHNICAL FIELD

The present invention relates to a method for producing a formate and a method for producing formic acid.

### BACKGROUND ART

As a technique for addressing problems of global warming and depletion of fossil fuels, high expectations are placed on a technique for converting carbon dioxide to useful compounds and a technique for utilizing hydrogen as next-generation energy.

Formic acid is an excellent compound as a hydrogen or carbon dioxide storage material since energy required for dehydrogenation is low and handling is easy, and can be utilized for the above-described techniques. For example, formic acid can be produced from formate obtained by reaction between hydrogen and a compound (compound C) such as carbon dioxide, hydrogencarbonate, or carbonate. As one example, Patent Literatures 1 and 2 disclose a method for producing formic acid from formate by using electrodialysis.

### CITATION LIST

### Patent Literature

Patent Literature 1: JP H07-299333A
Patent Literature 2: JP H10-036310A

### SUMMARY OF INVENTION

### Technical Problem

As described above, for example, formate as a precursor of formic acid can be produced by reaction between hydrogen and the above-described compound C. For example, this reaction can be performed in a two-phase system in which an organic solvent (organic phase) and an aqueous solvent (aqueous phase) are separate, by using a metal catalyst. In the reaction in the two-phase system, the generated formate is dissolved in the aqueous phase, and the metal catalyst is dissolved in the organic phase. By separating the aqueous phase and the organic phase, the formate and the metal catalyst can be easily separated. In the reaction in the two-phase system, an aqueous phase in which the concentration of formate is high can be advantageously produced with ease.

According to the examination performed by the inventors of the present invention, in the above-described reaction in the two-phase system, there is room for improvement in a catalyst turnover number (TON: Turnover Number) of the metal catalyst. In a case where the TON can be improved, formate can be more efficiently produced.

Therefore, an object of the present invention is to provide a method for producing a formate, which is suitable for improving a TON of a metal catalyst.

### Solution to Problem

The inventors of the present invention have found, as a result of thorough study, that a TON of a metal catalyst varies depending on not only a kind of a metal catalyst but also various reaction conditions such as a concentration of the metal catalyst, a concentration of a ligand, an amount of the compound C to be used, pressure of hydrogen, a reaction temperature, and a reaction time. The inventors of the present invention have further advanced the examination based on the findings, and have found anew a prediction formula for predicting a TON of a metal catalyst according to the reaction conditions, and have completed the present invention.

The present invention provides a method for producing a formate through reaction between hydrogen, and a compound C including at least one selected from the group consisting of carbon dioxide, hydrogencarbonate, and carbonate in the presence of a solvent by using a metal catalyst, and, in the method,
the solvent includes an organic solvent and an aqueous solvent,
a ligand is added to the solvent as necessary,
the reaction between hydrogen and the compound C is performed in a two-phase system in which the organic solvent and the aqueous solvent are separate under a condition that a value of y calculated by Equation (I) indicated below is more than 5.2,

$$y = 0.00398 {x}_{1} + ( - 0.0598 ) {x}_{2} + ( - 15.1 ) {x}_{3} + 6 \text{.66} {x}_{4} + ( - 0.00340 ) {x}_{5} + ( - 157 \; 99 ) {x}_{6} + 3.43 \times 1 {0}^{- 8} {x}_{7} + 4 \text{.97} , \qquad ( I )$$

in Equation (I), x₁ represents a reciprocal 1/a of a ratio a (mmol/L) of a substance amount of the metal catalyst to a volume of the organic solvent,
x₂ represents a ratio b of a substance amount of the ligand to the substance amount of the metal catalyst,
x₃ represents a value a/c obtained by dividing the ratio a (mmol/L) by a ratio c (mol/L) of a substance amount of the compound C that can be used for the reaction, to a volume of the aqueous solvent,
x₄ represents a value a×b obtained by multiplying the ratio a by the ratio b,
x₅ represents a ratio d of a reaction temperature (° C.) to a pressure (MPa) of the hydrogen,
x₆ represents a value a/e obtained by dividing the ratio a (mmol/L) by a catalyst turnover number e of the metal catalyst which is calculated in a test described below, and
x₇ represents a value e×f obtained by multiplying the catalyst turnover number e by a reaction time f (h),
Test: 1 ml of water and 5 mmol of potassium hydrogencarbonate are added into a vial having a stirring rod in an inert gas atmosphere, and 1 mL of toluene, 0.12 μmol of the metal catalyst, and 54 μmol of methyltrioctylammonium chloride are further added, the vial is set in an autoclave, the autoclave is sealed, a mixture in the vial is heated to 90° C. while being stirred, the autoclave is pressurized to 4.5 MPa with hydrogen when the temperature of the mixture has reached 90° C., the mixture is further stirred for 18 hours, the mixture is cooled, the vial is taken out from the autoclave, a substance amount of potassium formate included in the mixture is quantified, and the catalyst turnover number e of the metal catalyst is calculated from the obtained value.

Furthermore, the present invention provides a method for producing formic acid, and the method includes: producing the formate by the above-describe method for producing a formate; and protonating at least a part of the formate to generate formic acid.

### Advantageous Effects of Invention

The present invention can provide the method for producing a formate, which is suitable for improving a TON of the metal catalyst.

### BRIEF DESCRIPTION OF THE DRAWINGS

FIG. 1 is a schematic diagram illustrating one example of a three-chamber type electrodialyzer.

FIG. 2 is a general schematic diagram illustrating one example of a system for producing formic acid.

### DESCRIPTION OF EMBODIMENTS

A method for producing a formate according to a first aspect of the present invention is directed to a method for producing a formate through reaction between hydrogen, and a compound C including at least one selected from the group consisting of carbon dioxide, hydrogencarbonate, and carbonate in the presence of a solvent by using a metal catalyst, and, in the method,
the solvent includes an organic solvent and an aqueous solvent,
a ligand is added to the solvent as necessary,
the reaction between hydrogen and the compound C is performed in a two-phase system in which the organic solvent and the aqueous solvent are separate under a condition that a value of y calculated by Equation (I) indicated below is more than 5.2,

$$y = 0.00398 {x}_{1} + ( - 0.0598 ) {x}_{2} + ( - 15.1 ) {x}_{3} + 6 \text{.66} {x}_{4} + ( - 0.00340 ) {x}_{5} + ( - 157 \; 99 ) {x}_{6} + 3.43 \times 1 {0}^{- 8} {x}_{7} + 4 \text{.97} , \qquad ( I )$$

in Equation (I), x₁ represents a reciprocal 1/a of a ratio a (mmol/L) of a substance amount of the metal catalyst to a volume of the organic solvent,
x₂ represents a ratio b of a substance amount of the ligand to the substance amount of the metal catalyst,
x₃ represents a value a/c obtained by dividing the ratio a (mmol/L) by a ratio c (mol/L) of a substance amount of the compound C that can be used for the reaction, to a volume of the aqueous solvent,
x₄ represents a value a×b obtained by multiplying the ratio a by the ratio b,
x₅ represents a ratio d of a reaction temperature (° C.) to a pressure (MPa) of the hydrogen,
x₆ represents a value a/e obtained by dividing the ratio a (mmol/L) by a catalyst turnover number e of the metal catalyst which is calculated in a test described below, and
x₇ represents a value e×f obtained by multiplying the catalyst turnover number e by a reaction time f (h),

Test: 1 mL of water and 5 mmol of potassium hydrogencarbonate are added into a vial having a stirring rod in an inert gas atmosphere, and 1 mL of toluene, 0.12 μmol of the metal catalyst, and 54 μmol of methyltrioctylammonium chloride are further added, the vial is set in an autoclave, the autoclave is sealed, a mixture in the vial is heated to 90° C. while being stirred, the autoclave is pressurized to 4.5 MPa with hydrogen when the temperature of the mixture has reached 90° C., the mixture is further stirred for 18 hours, the mixture is cooled, the vial is taken out from the autoclave, a substance amount of potassium formate included in the mixture is quantified, and the catalyst turnover number e of the metal catalyst is calculated from the obtained value.

According to a second aspect of the present invention, for example, in the method for producing a formate according to the first aspect, the value of y is 5.7 or more.

According to a third aspect of the present invention, for example, in the method for producing a formate according to the first or the second aspect, a value of the x₁ is 100 or more.

According to a fourth aspect of the present invention, for example, in the method for producing a formate according to any one of the first to the third aspects, the value of x₃ is 0.002 or less.

According to a fifth aspect of the present invention, for example, in the method for producing a formate according to any one of the first to the fourth aspects, the value of x₄ is 0.028 or more.

According to a sixth aspect of the present invention, for example, in the method for producing a formate according to any one of the first to the fifth aspects, a value of the x₅ is 20 or less.

According to a seventh aspect of the present invention, for example, in the method for producing a formate according to any one of the first to the sixth aspects, the value of x₆ is 4.00×10⁻⁷ or less.

According to an eighth aspect of the present invention, for example, in the method for producing a formate according to any one of the first to the seventh aspects, the value of x₇ is 500,000 or more.

According to a ninth aspect of the present invention, for example, in the method for producing a formate according to any one of the first to the eighth aspects, the metal catalyst includes at least one selected from the group consisting of ruthenium and iridium.

According to a tenth aspect of the present invention, for example, in the method for producing a formate according to any one of the first to the ninth aspects, the metal catalyst includes at least one selected from the group consisting of a ruthenium complex represented by General formula (1) indicated below, a tautomer of the ruthenium complex, a stereoisomer of the ruthenium complex, and salt compounds thereof,

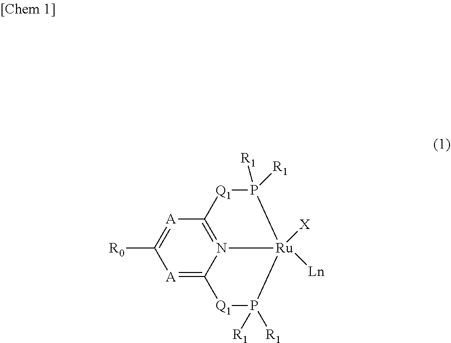

(in General formula (1), R₀ represents a hydrogen atom or an alkyl group,
each Q₁ independently represents CH₂, NH, or O,
each R₁ independently represents an alkyl group or an aryl group (in a case where Q₁ represents NH or O, at least one R₁ represents an aryl group),
each A independently represents CH, CR₅, or N,
R₅ represents an alkyl group, an aryl group, an aralkyl group, an amino group, a hydroxy group, or an alkoxy group,
X represents a halogen atom,
n represents 0 to 3, and
in a case where the number of Ls is plural, each L independently represents a neutral or anionic ligand).

According to an eleventh aspect of the present invention, for example, in the method for producing a formate according to any one of the first to the tenth aspects, the organic solvent includes toluene.

According to a twelfth aspect of the present invention, for example, in the method for producing a formate according to any one of the first to the eleventh aspects, the compound C includes potassium hydrogencarbonate.

According to a thirteenth aspect of the present invention, for example, in the method for producing a formate according to any one of the first to the twelfth aspects, a quaternary ammonium salt is further used as a phase transfer catalyst in the reaction.

A method for producing formic acid according to a fourteenth aspect of the present invention, includes:
producing the formate by the method for producing a formate according to any one of the first to the thirteenth aspects; and
protonating at least a part of the formate to generate formic acid.

The present invention will be described below in detail. However, the following description is not intended to restrict the present invention to a specific embodiment.

### Method for Producing Formate

A method for producing a formate according to the present embodiment is a method for producing a formate through reaction between hydrogen, and a compound C including at least one selected from the group consisting of carbon dioxide, hydrogencarbonate, and carbonate in the presence of a solvent by using a metal catalyst. The solvent includes an organic solvent and an aqueous solvent. To the solvent, a ligand is added as necessary. The reaction between hydrogen and the compound C is performed in a two-phase system in which the organic solvent and the aqueous solvent are separate under a condition that a value of y calculated by the following Equation (I) is more than 5.2. In the description herein, in the two-phase system, a phase including the organic solvent may be referred to as organic phase, and a phase including the aqueous solvent may be referred to as aqueous phase. The organic phase and the aqueous phase may be collectively referred to as reaction solution.

$$y = 0.00398 {x}_{1} + ( - 0.0598 ) {x}_{2} + ( - 15.1 ) {x}_{3} + 6 \text{.66} {x}_{4} + ( - 0.00340 ) {x}_{5} + ( - 157 \; 99 ) {x}_{6} + 3.43 \times 1 {0}^{- 8} {x}_{7} + 4.97 \qquad ( I )$$

In Equation (I), x₁ represents a reciprocal 1/a of a ratio a (mmol/L) of a substance amount of the metal catalyst to a volume of the organic solvent. The value of x₁ is, but is not particularly limited to, for example, 100 or more, and may be 130 or more, 150 or more, 180 or more, 200 or more, or 230 or more, and furthermore, may be 250 or more. The larger the x₁ is, the lower the concentration of the metal catalyst in the organic phase is and the higher the TON of the metal catalyst tends to become. The upper limit value of x₁ is, but is not particularly limited to, for example, 500, and may be 300.

x₂ represents a ratio b of a substance amount of the ligands added to the solvent as necessary to a substance amount of the metal catalyst. The value of x₂ is, but is not particularly limited to, for example, 0 to 20. The value of x₂ being 0 means that no ligands are added to the solvent. A ligand is preferably added to the solvent. From this viewpoint, the value of x₂ is 1 or more, 3 or more, or 5 or more, and furthermore, may be 8 or more.

x₃ represents a value a/c obtained by dividing the ratio a (mmol/L) by a ratio c (mol/L) of a substance amount of the compound C that can be used for the reaction, to a volume of the aqueous solvent. In the description herein, the compound C that can be used for the reaction specifically means the compound C dissolved in the reaction solution. The compound C may be dissolved over time in the reaction solution during the reaction depending on a method for introducing the compound C. In this case, the compound C dissolved in the reaction solution during the reaction is also regarded as the compound C that can be used for the reaction. As one example, in a case where carbon dioxide is introduced into a reaction vessel over time, the carbon dioxide is dissolved over time in the reaction solution while forming a carbonate with a base included in the reaction solution. By a reaction between the carbonate and hydrogen, formate is formed. The total substance amount of carbon dioxide dissolved in the reaction solution can be calculated from a substance amount of a base in the reaction solution, and the like.

The value of the ratio c is, but is not particularly limited to, for example, 0.1 mol/L or more, and may be 0.5 mol/L or more, 1 mol/L or more, or 3 mol/L or more, and furthermore, may be 5 mol/L or more. The upper limit value of the ratio c is, but is not particularly limited to, for example, 20 mol/L.

The value of x₃ is, but is not particularly limited to, for example, 0.002 or less, and may be 0.0018 or less, 0.0015 or less, or 0.0013 or less, and furthermore, may be 0.001 or less. The less the x₃ is, the higher the TON of the metal catalyst tends to become. The lower limit value of the x₃ is, but is not particularly limited to, for example, 0.0001.

x₄ represents a value a×b obtained by multiplying the ratio a by the ratio b. The value of x₄ is, but is not particularly limited to, for example, 0 to 0.1. The value of x₄ being 0 means that no ligands are added to the solvent. The value of x₄ may be 0.01 or more, 0.02 or more, 0.025 or more, 0.028 or more, 0.03 or more, or 0.033 or more, and furthermore, may be 0.035 or more. Particularly, in a case where a condition is set such that the value of x₄ is 0.028 or more, the TON of the metal catalyst tends to be enhanced while a relatively high yield is maintained.

x₅ represents a ratio d of a reaction temperature (C) to a pressure (MPa) of hydrogen in the reaction (hydrogenation of the compound C) between hydrogen and the compound C. The value of x₅ is, but is not particularly limited to, for example, 22 or less, and may be 21 or less or 20 or less, and furthermore, may be 19 or less. The less the x₅ is, the higher the TON of the metal catalyst tends to become. The lower limit value of x₅ is, but is not particularly limited to, for example, 1. The pressure of hydrogen means a pressure of hydrogen in the form of gas in a reaction vessel. The reaction temperature means a temperature of the reaction solution during the reaction.

x₆ represents a value a/e obtained by dividing the ratio a (mmol/L) by a catalyst turnover number e of the metal catalyst which is calculated in the following test.

Test: 1 ml of water and 5 mmol of potassium hydrogencarbonate (KHCO₃) are added in a vial having a stirring rod in an inert gas atmosphere, and 1 mL of toluene, 0.12 μmol of the metal catalyst, and 54 μmol of methyltrioctylammonium chloride are further added. The vial is set in an autoclave, and the autoclave is sealed. The mixture in the vial is heated to 90° C. while being stirred. When the temperature of the mixture has reached 90° C., the autoclave is pressurized to 4.5 MPa with hydrogen, and the mixture is further stirred for 18 hours. The mixture is cooled, and the vial is taken out from the autoclave. A substance amount of potassium formate included in the mixture is quantified, and the catalyst turnover number e of the metal catalyst is calculated from the obtained value.

In the above-described test, a vial made of glass can be used as the vial. The operation of adding the raw materials into the vial is, for example, performed in a glove box. In the above-described test, reaction between KHCO₃ as the compound C and hydrogen progresses in the two-phase system in which water and toluene are separate, to generate potassium formate (HCO₂K). Methyltrioctylammonium chloride functions as a phase transfer catalyst. The mixture can be cooled after the reaction by using, for example, an ice bath. When the vial is taken out from the autoclave, pressure in the autoclave needs to be carefully released.

A substance amount of the potassium formate included in the mixture can be quantified by, for example, the following method. Firstly, by placing the vial gently, the organic phase is located at an upper layer and the aqueous phase is located at a lower layer in the mixture. The upper layer and the lower layer are separated from each other, and the upper layer is removed. Thus, A g of the aqueous phase (aqueous solution) as the lower layer is obtained. In the aqueous solution, potassium formate and unreacted KHCO₃ are included in general. Subsequently, a part of the aqueous solution (B g of the aqueous solution) is dissolved in 500 μL of Deuterium oxide, and W g of dimethyl sulfoxide is additionally added as an internal standard, to produce a measurement sample. For the measurement sample, ¹H NMR measurement is performed. From the obtained NMR spectrum, an integral value la of a peak derived from the potassium formate, and an integral value Ib of a peak derived from the dimethyl sulfoxide are specified. A substance amount X (mol) of the potassium formate can be calculated according to the following Equation (i) based on these integral values and the like.

$$X = ( W / M ) \times ( Ia \times Ib / R ) \times ( A / B ) \qquad ( i )$$

(in Equation (i), W represents a weight (g) of the dimethyl sulfoxide used for quantifying the potassium formate,
M represents a molecular weight of the dimethyl sulfoxide,
R represents a ratio of the number of protons of the dimethyl sulfoxide per one molecule, to the number of protons of the potassium formate per one molecule,
la represents an integral value of the NMR peak derived from the potassium formate,
lb represents an integral value of the NMR peak derived from the dimethyl sulfoxide,
A represents a weight (g) of the aqueous phase (aqueous solution) obtained in the above-described test, and
B represents a weight (g) of the aqueous solution used for quantifying the potassium formate.)

Furthermore, the catalyst turnover number e of the metal catalyst can be calculated according to the following Equation (ii) based on the substance amount X (mol) of the potassium formate which is calculated according to Equation (i).

$$Catalyst turnover number e = X / Y \qquad ( ii )$$

(in Equation (ii), X represents a substance amount (mol) of the potassium formate which is calculated according to Equation (i), and
Y represents a substance amount (mol) of the metal catalyst used in the above-described test.)

The catalyst turnover number e can be used as an index of catalytic activity of the metal catalyst in the reaction for synthesizing the formate. The catalyst turnover number e is, but is not particularly limited to, for example, 1,000 or more, and may be 4,000 or more, 6,000 or more, 10,000 or more, 15,000 or more, or 20,000 or more, and furthermore, may be 25,000 or more. The upper limit value of the catalyst turnover number e is, but is not particularly limited to, for example, 1,000,000 and may be 100,000.

The value of x₆ is, but is not particularly limited to, for example, 4.00×10⁻⁷ or less, and may be 3.50×10⁻⁷ or less, 3.00×10⁻⁷ or less, 2.50×10⁻⁷ or less, or 2.00×10⁻⁷ or less, and furthermore, may be 1.50×10⁻⁷ or less. The less the x₆ is, the higher the TON of the metal catalyst tends to become. The lower limit value of x₆ is, but is not particularly limited to, for example, 1.00×10⁻⁸.

x₇ represents a value e×f obtained by multiplying the catalyst turnover number e by the reaction time f (h). The value of x₇ is, but is not particularly limited to, for example, 500,000 or more, and may be 800,000 or more, 1,000,000 or more, 1,200,000 or more, or 1,300,000 or more, and furthermore, may be 1,500,000 or more. The larger the x₇ is, the higher the TON of the metal catalyst tends to become. The upper limit value of x₇ is, but is not particularly limited to, for example, 10,000,000 and may be 5,000,000.

The value of y calculated by Equation (I) is preferably 5.4 or more, and may be 5.5 or more, 5.6 or more, or 5.65 or more, and furthermore, may be 5.7 or more. The upper limit value of y is, but is not particularly limited to, for example, 10, and may be 8, 7, or 6. The value of y represents an index of the TON of the metal catalyst for a case where hydrogen and the compound C are caused to react under a specific reaction condition, and tends to conform well to a common logarithm (log (TON)) of the TON.

Equation (I) is generated by the following method. Firstly, various reaction conditions such as a kind of the metal catalyst, a concentration of the metal catalyst, a concentration of the ligand, an amount of the compound C to be used, pressure of hydrogen, a reaction temperature, and a reaction time are discretionarily set, and reaction between hydrogen and the compound C is performed. Thus, 70 or more pieces of experimental data in which the reaction conditions and the TON of the metal catalyst are associated with each other are obtained. Subsequently, various explanatory variables are generated based on the obtained experimental data, and Lasso (least absolute shrinkage and selection operator) regression is performed. The Lasso regression is a linear regression technique having an L1 regularization term. Explanatory variables having high degrees of importance are extracted by the Lasso regression to obtain Equation (I).

The method for producing the formate includes, for example, a step of causing hydrogen and the compound C to react with each other in the presence of a solvent by using the metal catalyst to generate formate in the reaction solution. In the description herein, this step may be referred to as first step. In the first step, hydrogen and the compound C are caused to react with each other in the two-phase system in which the organic solvent and the aqueous solvent are separate, as described above. In this reaction, the metal catalyst is, for example, dissolved in the organic phase. The formate generated by the reaction is dissolved in the aqueous phase. Thus, the reaction for generating the formate can be inhibited from halting due to equilibrium, and the formate can be generated at a high yield. Furthermore, the aqueous phase and the organic phase can be separated by a simple method. Therefore, an expensive metal catalyst tends to be reused without deactivating catalytic activity. By reusing the metal catalyst, high productivity can be achieved.

In the first step, hydrogen and carbon dioxide can be stored as formate (for example, alkali metal formate). Formate has a high hydrogen storage density, is safe, and stable as a chemical substance, so that formate can be easily handled, and hydrogen and carbon dioxide can be advantageously stored for a long time period. Formate has high solubility with respect to an aqueous solvent, and can be fractionated as an aqueous solution of formate which has a high concentration. After the concentration of formate is adjusted as necessary, the aqueous solution of the formate can be supplied to a formic acid producing step described below.

For example, the first step can be performed as follows. Firstly, a reaction vessel having a stirring device is prepared, and a solvent is introduced into the reaction vessel. A phase transfer catalyst may be further added as necessary. The metal catalyst is added into the reaction vessel, and dissolved in the solvent to prepare a catalyst solution. Hydrogen and the compound C are introduced into the reaction vessel, to cause a reaction.

### (Solvent)

The solvent is not particularly limited as long as the solvent can form a two-phase system in which the organic solvent and the aqueous solvent are present in a separated state, and preferably includes a solvent in which the metal catalyst is uniformly dissolved.

Examples of the aqueous solvent include water, methanol, ethanol, ethylene glycol, glycerin, and mixed solvents thereof. Water is preferred from the viewpoint of a low load on the environment.

Examples of the organic solvent include toluene, benzene, xylene, propylene carbonate, dioxane, dimethyl sulfoxide, tetrahydrofuran, ethyl acetate, methyl cyclohexane, cyclopentyl methyl ether, and mixed solvents thereof, and the organic solvent preferably includes toluene or dioxane and more preferably includes toluene from the viewpoint of separability from the aqueous solvent.

### (Metal Catalyst)

The metal catalyst is a compound (metal element compound) that contains a metal element, and is preferably a metal complex catalyst. For example, the metal complex catalyst has a metal element and a ligand coordinated to the metal element. Furthermore, the metal catalyst is preferably dissolved in the organic solvent.

Examples of the metal element compound include: salts of metal elements with inorganic acids, such as a hydride salt, an oxide salt, a halide salt (chloride salt or the like), a hydroxide salt, a carbonic acid salt, a hydrogen carbonic acid salt, a sulfuric acid salt, a nitric acid salt, a phosphoric acid salt, a boric acid salt, a halogen acid salt, a perhalogen acid salt, a halous acid salt, a hypohalous acid salt, and a thiocyanic acid salt; salts of metal elements with organic acids, such as an alkoxide salt, a carboxylic acid salt (acetic acid salt, (meth)acrylic acid salt, or the like), and a sulfonic acid salt (trifluoromethanesulfonic acid salt or the like); salts of metal elements with organic bases, such as an amide salt, a sulfonamide salt, and a sulfonimide salt (bis(trifluoromethanesulfonyl)imide salt or the like); complex salts such as an acetylacetone salt, a hexafluoroacetylacetone salt, a porphyrin salt, a phthalocyanine salt, and a cyclopentadiene salt; and complexes or salts containing one or more of nitrogen compounds including a linear amine, a cyclic amine, an aromatic amine, or the like, phosphorus compounds, compounds containing phosphorus and nitrogen, sulfur compounds, carbon monoxide, carbon dioxide, and water. These compounds may be either hydrates or anhydrides and are not particularly limited. Among them, a halide salt, a complex containing a phosphorus compound, a complex containing a nitrogen compound, and a complex or salt containing a compound containing phosphorus and nitrogen are preferred from the viewpoint of further enhancing the formic aid generating efficiency. One of them may be used alone, or two or more of them may be used in combination.

As the metal element compound, a commercially available one can be used, and a metal element compound produced by a known method or the like can be used. Examples of the known method include the method described in JP5896539B and the methods described in Chem. Rev. 2017, 117, 9804-9838 and Chem. Rev. 2018, 118, 372-433.

The metal catalyst preferably includes at least one selected from the group consisting of ruthenium, iridium, iron, nickel, and cobalt, more preferably includes at least one selected from the group consisting of ruthenium and iridium, and even more preferably includes ruthenium.

Particularly, the metal catalyst preferably includes at least one selected from the group consisting of a ruthenium complex represented by the following General formula (1), a tautomer of the ruthenium complex, a stereoisomer of the ruthenium complex, and salt compounds thereof. The ruthenium complex represented by General formula (1) is dissolved in the organic solvent and is not dissolved in water in general. Formate generated by the reaction is easily dissolved in water. Therefore, the ruthenium complex allows the metal catalyst and formate to be easily separated after the reaction in the two-phase system, and the metal catalyst and formate can be easily separated and individually collected from the reaction system. By the ruthenium complex, formate also tends to be produced at a high yield. In the production method of the present embodiment, the formate generated by the reaction and the metal catalyst can be separated by a simple operation, and an expensive metal catalyst can be reused.

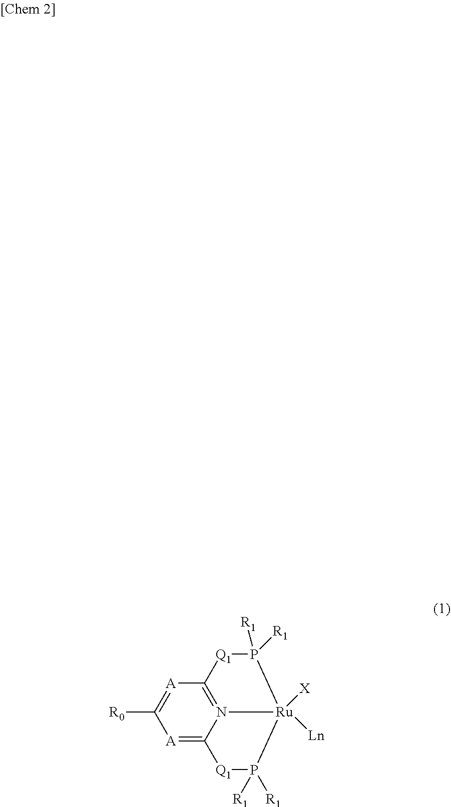

(In General formula (1), R₀ represents a hydrogen atom or an alkyl group, each Q₁ independently represents CH₂, NH, or O,
each R₁ independently represents an alkyl group or an aryl group (in a case where Q₁ represents NH or O, at least one R₁ represents an aryl group),
each A independently represents CH, CR₅, or N,
R₅ represents an alkyl group, an aryl group, an aralkyl group, an amino group, a hydroxy group, or an alkoxy group,
X represents a halogen atom,
n represents 0 to 3, and
each L independently represents a neutral or anionic ligand in a case where the number of Ls is plural.)

In General formula (1), R₀ represents a hydrogen atom or an alkyl group. The alkyl group represented by R₀ is, for example, a linear, branched, or cyclic substituted or unsubstituted alkyl group. For example, the alkyl group represented by R₀ is preferably a C1 to C30 alkyl group, such as a methyl group, an ethyl group, an n-propyl group, an i-propyl group, a t-butyl group, an n-octyl group, an eicosyl group, and a 2-ethylhexyl group. An alkyl group in which the number of carbon atoms is 6 or less is preferable from the viewpoint of easily obtaining a raw material, and a methyl group is preferable. R₀ in General formula (1) preferably represents a hydrogen atom or a methyl group.

Each R₁ in General formula (1) independently represents an alkyl group or an aryl group. However, in a case where Q₁ represents NH or O, at least one R₁ represents an aryl group. The alkyl group represented by R₁ is, for example, a linear, branched, or cyclic substituted or unsubstituted alkyl group. The alkyl group represented by R₁ is preferably a C1 to C30 alkyl group, such as a methyl group, an ethyl group, an n-propyl group, an i-propyl group, a t-butyl group, an n-octyl group, an eicosyl group, and a 2-ethylhexyl group. An alkyl group in which the number of carbon atoms is 12 or less is preferable from the viewpoint of catalytic activity, and a t-butyl group is preferable.

The aryl group represented by R₁ is a C6 to C30 substituted or unsubstituted aryl group, such as a phenyl group, a p-tolyl group, a naphthyl group, an m-chlorophenyl group, and an o-hexadecanoylaminophenyl group. An aryl group in which the number of carbon atoms is 12 or less is preferable, and a phenyl group is more preferable.

Each A independently represents CH, CR₅, or N, and R₅ represents an alkyl group, an aryl group, an aralkyl group, an amino group, a hydroxy group, or an alkoxy group. The alkyl group represented by R₅ is, for example, a linear, branched, or cyclic substituted or unsubstituted alkyl group. The alkyl group represented by R₅ is preferably a C1 to C30 alkyl group, such as a methyl group, an ethyl group, an n-propyl group, an i-propyl group, a t-butyl group, an n-octyl group, an eicosyl group, and a 2-ethylhexyl group. An alkyl group in which the number of carbon atoms is 12 or less is preferable from the viewpoint of easily obtaining a raw material, and a methyl group is preferable.

The aryl group represented by R₅ is, for example, a C6 to C30 substituted or unsubstituted aryl group, such as a phenyl group, a p-tolyl group, a naphthyl group, an m-chlorophenyl group, or an o-hexadecanoylaminophenyl group, and an aryl group in which the number of carbon atoms is 12 or less is preferable, and a phenyl group is more preferable.

The aralkyl group represented by R₅ is, for example, a substituted or unsubstituted aralkyl group in which the number of carbon atoms is 30 or less, and examples thereof include a trityl group, a benzyl group, a phenethyl group, a tritylmethyl group, a diphenylmethyl group, and a naphthylmethyl group, and an aralkyl group in which the number of carbon atoms is 12 or less is preferable.

The alkoxy group represented by R₅ is preferably a C1 to C30 substituted or unsubstituted alkoxy group, such as a methoxy group, an ethoxy group, an isopropoxy group, a t-butoxy group, an n-octyloxy group, and a 2-methoxyethoxy group.

X represents a halogen atom, and preferably represents a chlorine atom.

n represents an integer of 0 to 3, and represents the number of ligands coordinated to ruthenium. n is preferably 2 or 3 from the viewpoint of stability of the catalyst.

In a case where the number of Ls is plural, each L independently represents a neutral or anionic ligand. Examples of the neutral ligand represented by L include ammonia, carbon monoxide, phosphines (for example, triphenylphosphine, tris(4-methoxyphenyl)phosphine), phosphine oxides (for example, triphenyl phosphine oxide), sulfides (for example, dimethyl sulfide), sulfoxides (for example, dimethyl sulfoxide), ethers (for example, diethyl ether), nitriles (for example, p-methylbenzonitrile), and heterocyclic compounds (for example, pyridine, N,N-dimethyl-4-aminopyridine, tetrahydrothiophene, tetrahydrofuran), and the neutral ligand is preferably triphenylphosphine. Examples of the anionic ligand represented by L include a hydride ion (hydrogen atom), a nitrate ion, and a cyanide ion, and the anionic ligand is preferably a hydride ion (hydrogen atom).

In General formula (1), A may represent CH, and Q₁ may represent NH. Preferably, n represents 1 to 3, and each L independently represents a hydrogen atom, carbon monoxide, or triphenylphosphine.

One kind of the ruthenium complex represented by General formula (1) may be used alone, or two or more of the ruthenium complexes may be used in combination.

The ruthenium complex represented by General formula (1) may be a ruthenium complex represented by the following General formula (3).

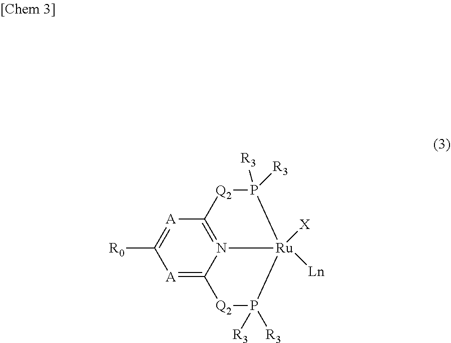

(In General formula (3), R₀ represents a hydrogen atom or an alkyl group,
each Q₂ independently represents NH or O,
each R₃ independently represents an aryl group,
each A independently represents CH, CR₅, or N,
R₅ represents an alkyl group, an aryl group, an aralkyl group, an amino group, a hydroxy group, or an alkoxy group,
X represents a halogen atom,
n represents 0 to 3, and
each L independently represents a neutral or anionic ligand in a case where the number of Ls is plural.)

R₀, A, R₅, X, n, and L in General formula (3) are equivalent to R₀, A, R₅, X, n, and L, respectively, in General formula (1), and the preferable ranges are also the same.

The aryl group represented by each R₃ in General formula (3) is equivalent to the aryl group represented by each R₁ in General formula (1), and the preferable ranges are also the same.

As the ruthenium complexes represented by General formula (1) and General formula (3), a ruthenium complex produced by a known method or the like can be used. Examples of the known method include a method described in, for example, E. Pidko et al., ChemCatChem 2014, 6, 1526-1530.

In the ruthenium complexes represented by General formula (1) and General formula (3), a stereoisomer may be generated due to a coordination form or conformation of the ligands. The metal catalyst may be a mixture of the stereoisomers, or may be a pure single isomer.

Specific examples of the ruthenium complex represented by General formula (1), the ruthenium complex represented by General formula (3), and the ligands included in these complexes include compounds described below. In the compounds illustrated below, tBu represents a tertiary butyl group, and Ph represents a phenyl group.

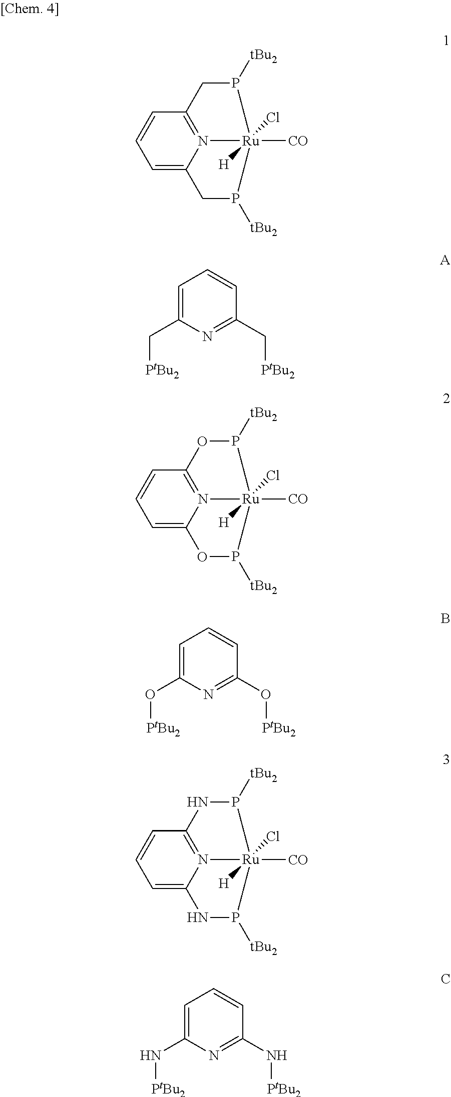

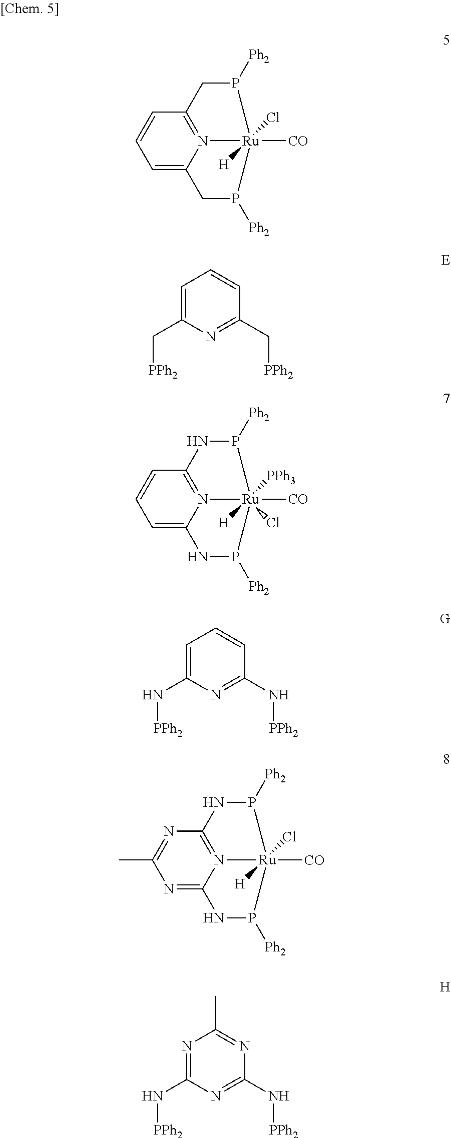

An amount of the metal catalyst (preferably, ruthenium complex) to be used is not particularly limited as long as the above-described value of y is more than 5.2. The amount of the metal catalyst to be used is preferably 0.1 μmol or more, more preferably 0.5 μmol or more, and even more preferably 1 μmol or more with respect to 1 L of the solvent from the viewpoint of sufficiently exhibiting the function of the metal catalyst. The amount of the metal catalyst to be used is preferably 1 mol or less, more preferably 10 mmol or less, and even more preferably 1 mmol or less with respect to 1 L of the solvent from the viewpoint of cost. Furthermore, the amount of the metal catalyst to be used may be 100 μmol or less or 10 μmol or less with respect to 1 L of the solvent from the viewpoint of enhancing the TON of the metal catalyst. In a case where two or more kinds of the metal catalysts are used, the total amount of the metal catalysts to be used is in the above-described range.

### (Ligand)

As described above, in the first step, the ligand is added to the solvent as necessary. As one example, in a case where the metal catalyst is a metal complex catalyst, the same ligand as the ligand included in the metal complex catalyst may be added to the solvent. Thus, an excess amount of ligands of the metal complex catalyst can exist in the reaction solution. For example, in a case where the metal catalyst is the ruthenium complex represented by General formula (1), it is preferable that a ligand represented by the following General formula (4) is further added.

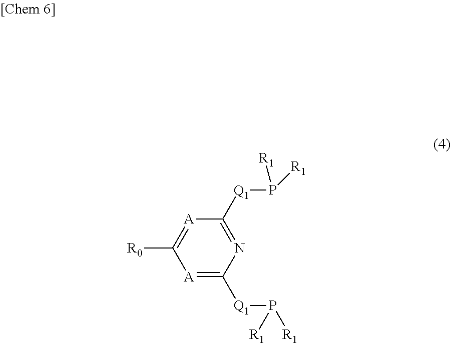

(In General formula (4), R₀ represents a hydrogen atom or an alkyl group,
each Q₁ independently represents CH₂, NH, or O,
each R₁ independently represents an alkyl group or an aryl group (in a case where Q₁ represents NH or O, at least one R₁ represents an aryl group),
each A independently represents CH, CR₅, or N, and
R₅ represents an alkyl group, an aryl group, an aralkyl group, an amino group, a hydroxy group, or an alkoxy group.)

R₀, Q₁, R₁, A, and R₅ in General formula (4) are equivalent to R₀, Q₁, R₁, A, and R₅, respectively, in General formula (1), and the preferable ranges are also the same.

In a case where the metal catalyst is the ruthenium complex represented by General formula (3), it is preferable that a ligand represented by the following General formula (5) is further added.

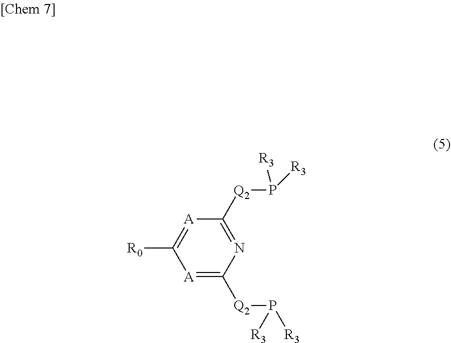

(In General formula (5), R₀ represents a hydrogen atom or an alkyl group,
each Q₂ independently represents NH or O,
each R₃ independently represents an aryl group,
each A independently represents CH, CR₅, or N, and
R₅ represents an alkyl group, an aryl group, an aralkyl group, an amino group, a hydroxy group, or an alkoxy group.)

R₀, Q₂, R₃, A, and R₅ in General formula (5) are equivalent to R₀, Q₂, R₃, A, and R₅, respectively, in General formula (3), and the preferable ranges are also the same.

In a case where the ligands forming the complex are excessively added to the reaction system, even if the ligands are oxidized and degraded due to oxygen or impurities included in the system, a catalyst function may be restored by exchange between the degraded ligands and the added ligands. Therefore, by adding the ligands, stability of the metal catalyst can be enhanced.

The ligand represented by General formula (4) or General formula (5) may be added to the reaction mixture when the reaction mixture is prepared. Although the ligand may be added during the reaction, the ligand is preferably added when the reaction mixture is prepared from the viewpoint of managing the process.

### (Phase Transfer Catalyst)

In the method for producing formic acid according to the present embodiment, the reaction needs to be performed in the two-phase system. Therefore, a phase transfer catalyst for smoothly transferring substances between the two phases, may be used. Examples of the phase transfer catalyst include a quaternary ammonium salt, a quaternary phosphate, a macrocyclic polyether such as a crown ether, a nitrogen-containing macrocyclic polyether such as a cryptand, a nitrogen-containing linear polyether, and polyethylene glycol and an alkyl ether thereof. Among them, a quaternary ammonium salt is preferred from the viewpoint of easily transferring substances between the aqueous solvent and the organic solvent even under a mild reaction condition. In other words, it is preferable to further use a quaternary ammonium salt as the phase transfer catalyst in the reaction between hydrogen and the compound C.

Examples of the quaternary ammonium salt include methyltrioctylammonium chloride, benzyltrimethylammonium chloride, trimethylphenylammonium bromide, tributylammonium tribromide, tetrahexylammonium hydrogen sulfate, decyltrimethylammonium bromide, diallyldimethylammonium chloride, dodecyltrimethylammonium bromide, dimethyldioctadecylammonium bromide, tetraethylammonium tetrafluoroborate, ethyltrimethylammonium iodide, tris(2-hydroxyethyl)methylammonium hydroxide, tetramethylammonium acetate, tetramethylammonium bromide, and tetraethylammonium iodide. Methyltrioctylammonium chloride is preferable.

An amount of the phase transfer catalyst to be used is not particularly limited as long as formate can be produced. The amount of the phase transfer catalyst to be used is preferably 0.1 mmol or more, more preferably 0.5 mmol or more, and even more preferably 1 mmol or more with respect to 1 L of the organic phase and the aqueous phase solvents such that the phase transfer catalyst acts to efficiently aid in transferring carbonate or hydrogencarbonate. The amount of the phase transfer catalyst to be used is preferably 1 mol or less, more preferably 500 mmol or less, and even more preferably 100 mmol or less with respect to 1 L of the organic phase and the aqueous phase solvents from the viewpoint of cost. In a case where two or more kinds of the phase transfer catalysts are used, the total amount of the phase transfer catalysts to be used is in the above-described range.

### (Carbon Dioxide and Hydrogen)

As hydrogen used in the present embodiment, either hydrogen in the form of gas from a gas cylinder or liquid hydrogen can be used. As a hydrogen supply source, for example, hydrogen generated in a smelting process in iron manufacture or hydrogen generated in a soda manufacturing process can be used. Hydrogen generated by electrolysis of water can also be utilized.

Carbon dioxide used in the present embodiment may be pure carbon dioxide gas, or may be a mixture with a component other than carbon dioxide. Mixed gas with another component may be prepared by individually introducing carbon dioxide gas and another gas, or mixed gas may be prepared in advance before being introduced. Examples of the component other than carbon dioxide include inert gases such as nitrogen and argon, water vapor, and any other component included in exhaust gas and the like. Examples of carbon dioxide include carbon dioxide in the form of gas from a gas cylinder, carbon dioxide in the form of liquid, supercritical carbon dioxide, and dry ice.

The hydrogen gas and the carbon dioxide gas may be individually introduced into the reaction system, or may be introduced as mixed gas. Hydrogen and carbon dioxide may be used at the same proportion on a mole basis. However, hydrogen is preferably used in excess.

In a case where hydrogen in the form of gas from a gas cylinder is used as hydrogen, the pressure of the hydrogen is, for example, 0.1 MPa or more, may be 0.2 MPa or more, 0.5 MPa or more, 1 MPa or more, 4 MPa or more, or 4.5 MPa or more, and furthermore, may be 5 MPa or more, from the viewpoint of sufficiently ensuring reactivity. The pressure of the hydrogen is preferably 50 MPa or less, more preferably 20 MPa or less, and even more preferably 10 MPa or less in order to address enlargement of facilities.

The pressure of the carbon dioxide is preferably 0.1 MPa or more, more preferably 0.2 MPa or more, and even more preferably 0.5 MPa or more from the viewpoint of sufficiently ensuring reactivity. The pressure of the carbon dioxide is preferably 50 MPa or less, more preferably 20 MPa or less, and even more preferably 10 MPa or less in order to address enlargement of facilities.

Hydrogen gas and carbon dioxide gas may be introduced into the catalyst solution by bubbling (blowing). Hydrogen gas and gas including carbon dioxide are introduced and thereafter stirred by a stirring device. By, for example, rotating a reaction vessel, the catalyst solution, and hydrogen gas and carbon dioxide gas may be stirred.

A method for introducing carbon dioxide, hydrogen, the metal catalyst, the solvent, and the like which are used for the reaction, into a reaction vessel, is not particularly limited. All of the raw materials and the like may be collectively introduced, a part or all of the raw materials and the like may be introduced stepwise, or a part or all of the raw materials and the like may be continuously introduced. An introduction method in which these methods are combined may be used.

### (Hydrogencarbonate and Carbonate)

Examples of hydrogencarbonate and carbonate used in the present embodiment include carbonate and hydrogencarbonate of an alkali metal or an alkaline-earth metal. Examples of hydrogencarbonate include sodium hydrogencarbonate and potassium hydrogencarbonate. Potassium hydrogencarbonate is preferable from the viewpoint of high solubility with respect to water. That is, in the present embodiment, the compound C preferably includes potassium hydrogencarbonate as hydrogencarbonate. Examples of carbonate include sodium carbonate, potassium carbonate, potassium sodium carbonate, and sodium sesquicarbonate.

Hydrogencarbonate and carbonate can be generated by reaction between a base and carbon dioxide. For example, hydrogencarbonate or carbonate may be generated by introducing carbon dioxide into a basic solution.

Examples of a solvent for the basic solution in generation of hydrogencarbonate or carbonate include, but are not particularly limited to, water, methanol, ethanol, N,N-dimethylformamide, dimethyl sulfoxide, tetrahydrofuran, benzene, toluene, and mixed solvents thereof, and the solvent preferably includes water, and is more preferably water. The base used for the basic solution is not particularly limited as long as the base can react with carbon dioxide to generate hydrogencarbonate or carbonate, and the base is preferably hydroxide. Examples of the base include lithium hydrogencarbonate, sodium hydrogencarbonate, potassium hydrogencarbonate, cesium hydrogencarbonate, potassium hydroxide, sodium hydroxide, diazabicycloundecene, and triethylamine. Among them, the base is preferably hydroxide, more preferably potassium hydroxide or sodium hydroxide, and even more preferably potassium hydroxide.

A content of the base in the basic solution is not particularly limited as long as hydrogencarbonate and carbonate can be produced. The content of the base is preferably 0.1 mol or more, more preferably 0.5 mol or more, and even more preferably 1 mol or more with respect to 1 L of the aqueous phase solvent from the viewpoint of ensuring an amount of generated formate. The content of the base is preferably 30 mol or less, more preferably 20 mol or less, and even more preferably 15 mol or less from the viewpoint of efficiency of the reaction. If the content of the base is greater relative to the solubility of the aqueous phase, the solution may be suspended.

A ratio between carbon dioxide and the base in terms of amount to be used in the reaction between carbon dioxide and the base is preferably 0.1 or more, more preferably 0.5 or more, and even more preferably 1.0 or more as a molar ratio from the viewpoint of generating carbonate from carbon dioxide. The ratio is preferably 8.0 or less, more preferably 5.0 or less, and even more preferably 3.0 or less from the viewpoint of utilization efficiency of carbon dioxide. The ratio between carbon dioxide and the base in terms of amount to be used may be a ratio between molar amounts of carbon dioxide and the base to be introduced into a reaction vessel, and is a molar amount (mol) of CO₂/a molar amount (mol) of the base. In a case where the ratio between carbon dioxide and the base in terms of amount to be used is in the above-described range, carbon dioxide is inhibited from being excessively charged into the reaction vessel, and an amount of unreacted carbon dioxide can be minimized, so that the final formic acid conversion efficiency is likely to be enhanced. In the same vessel, carbon dioxide can be hydrogenated through hydrogencarbonate or carbonate by reaction between carbon dioxide and the base to generate formate. Unreacted carbon dioxide can be collected from the reaction vessel and reused.

A method and an order for introducing carbon dioxide and the base into the reaction vessel are not particularly limited. However, carbon dioxide is preferably introduced after the base is introduced into the reaction vessel. One or both of carbon dioxide and the base may be continuously introduced or intermittently introduced.

In the reaction for generating hydrogencarbonate or carbonate by reaction between carbon dioxide and the base, the reaction temperature is, but is not particularly limited to, preferably 0° C. or higher, more preferably 10° C. or higher, and even more preferably 20° C. or higher in order to dissolve carbon dioxide in the aqueous phase. The reaction temperature is preferably 100° C. or lower, more preferably 80° C. or lower, and even more preferably 40° C. or lower.

In the reaction for generating hydrogencarbonate or carbonate by reaction between carbon dioxide and the base, a reaction time is, but is not particularly limited to, for example, preferably 0.5 hours or longer, more preferably one hour or longer, and even more preferably two hours or longer, from the viewpoint of sufficiently ensuring an amount of generated hydrogencarbonate or carbonate. The reaction time is preferably 24 hours or less, more preferably 12 hours or less, and even more preferably 6 hours or less from the viewpoint of cost.

Hydrogencarbonate and carbonate generated by reaction between carbon dioxide and the base can be used as the compound C that is to react with hydrogen. By generating hydrogencarbonate or carbonate by reaction between carbon dioxide and the base in the reaction vessel, hydrogencarbonate or carbonate may be introduced into the reaction vessel.

### (Reaction Condition)

In the method for producing the formate according to the present embodiment, a reaction condition (reaction condition in the first step) is not particularly limited as long as the above-described value of y is more than 5.2. In the present embodiment, the reaction condition may be changed as appropriate during the reaction depending on the case. However, the reaction condition preferably remains unchanged. The form of the reaction vessel used for the reaction is not particularly limited.

In the first step, for example, the reaction solution is stirred. The condition for stirring the reaction solution is not particularly limited. However, the stirring power is preferably 0.2 kW/m³ or more and more preferably 0.5 kW/m³ or more. The higher the stirring power is, the higher the dispersibility of gas into the aqueous phase and the organic phase tends to become. By stirring the reaction solution, gas (for example, hydrogen in the form of gas) is involved into the reaction solution from the upper portion of the liquid surface of the reaction solution, so that gas is filled into the aqueous phase and the organic phase. However, the method for filling gas into the aqueous phase and the organic phase is not limited to the above-described method, and a sparger may be used.

The shape of the stirring blade used for stirring the reaction solution is not particularly limited. Examples of the stirring blade include not only an anchor blade, a turbine blade, and a paddle blade but also a blade that is generically called large blade, such as FULLZONE (registered trademark) blade (Kobelco Eco-Solutions Co., Ltd.) and MAXBLEND (registered trademark) blade (Sumitomo Heavy Industries Process Equipment Co., Ltd.).

In the present embodiment, examples of the reaction between hydrogen and the compound C include a reaction between hydrogen and carbon dioxide, a reaction between hydrogen and hydrogencarbonate, and a reaction between hydrogen and carbonate. In the reaction between hydrogen and carbon dioxide, for example, a reaction in which carbon dioxide forms carbonate, and a reaction in which formate is generated by the carbonate and hydrogen simultaneously progress.

A method and an order for introducing hydrogen and the compound C into the reaction vessel are not particularly limited. For example, in the reaction between hydrogen and carbon dioxide, hydrogen and carbon dioxide are preferably introduced simultaneously. Hydrogen and carbon dioxide may be individually introduced, or may be introduced as mixed gas. One or both of hydrogen and carbon dioxide may be continuously introduced or intermittently introduced. In the reaction between hydrogen and hydrogencarbonate and the reaction between hydrogen and carbonate, hydrogen is preferably introduced after hydrogencarbonate or carbonate is introduced into the reaction vessel. One or both of hydrogen, and hydrogencarbonate or carbonate may be continuously introduced or intermittently introduced.

In the reaction between hydrogen and the compound C, a reaction temperature is not particularly limited, and is preferably 30° C. or higher, more preferably 40° C. or higher, and even more preferably 50° C. or higher in order to cause the reaction to efficiently progress. The reaction temperature is preferably 200° C. or lower, more preferably 150° C. or lower, and even more preferably 100° C. or lower from the viewpoint of energy efficiency. The reaction temperature can be adjusted by heating or cooling, and the temperature is preferably increased by heating. In the reaction between hydrogen and carbon dioxide, for example, the temperature may be increased by heating after hydrogen and carbon dioxide are introduced into the reaction vessel, or carbon dioxide is introduced into the reaction vessel and the temperature is increased, and thereafter, hydrogen may be introduced. In the reaction between hydrogen, and hydrogencarbonate or carbonate, for example, it is preferable that hydrogencarbonate or carbonate is introduced (generated) into the reaction vessel and the temperature is increased, and hydrogen is thereafter introduced.

In the reaction between hydrogen and the compound C, a reaction time is not particularly limited, and is, for example, 0.5 hours or longer, and may be one hour or longer, two hours or longer, six hours or longer, 12 hours or longer, 24 hours or longer, 36 hours or longer, or 48 hours or longer, and furthermore, may be 60 hours or longer from the viewpoint of sufficiently ensuring an amount of generated formate and enhancing the TON of the metal catalyst. The upper limit of the reaction time is, but is not particularly limited to, for example, 500 hours.

In the reaction between hydrogen and the compound C, a reaction pressure (pressure of gas in the reaction vessel) is, but is not particularly limited to, for example, 0.1 MPa or more, and may be 0.2 MPa or more, 0.5 MPa or more, 1 MPa or more, 4 MPa or more, or 4.5 MPa or more, and furthermore, may be 5 MPa or more from the viewpoint of enhancing the TON of the metal catalyst. The upper limit value of the reaction pressure is, but is not particularly limited to, for example, 50 MPa, and may be 20 MPa or 10 MPa.

A concentration (concentration of formate in the aqueous phase) of the formate generated in the first step is preferably 1 mol/L or more, more preferably 2.5 mol/L or more, and even more preferably 5 mol/L or more in order to produce formate at a high yield with excellent productivity. The concentration of formate is preferably 30 mol/L or less, more preferably 25 mol/L or less, and even more preferably 20 mol/L or less in order to simplify the production process by producing formate in a state where the formate is dissolved.

In the present embodiment, a yield of formate by the reaction between hydrogen and the compound C is preferably sufficient for practical use. The yield is preferably 30% or more, and may be 40% or more, 50% or more, 55% or more, 60% or more, 65% or more, 70% or more, or 75% or more, and furthermore, may be 80% or more. The upper limit value of the yield is, but is not particularly limited to, for example, 99%.

The production method of the present embodiment is suitable for improving the TON of the metal catalyst in the reaction between hydrogen and the compound C. The TON of the metal catalyst in the reaction is, for example, 300,000 or more, and may be 400,000 or more, 500,000 or more, 550,000 or more, 600,000 or more, 650,000 or more, or 700,000 or more, and furthermore, may be 750,000 or more. The upper limit value of the TON of the metal catalyst is, but is not particularly limited to, for example, 5,000,000.

In the present embodiment, both a sufficient yield for practical use and high TON are preferably achieved in the reaction between hydrogen and the compound C. As one example, it is preferable that the yield is 40% or more and the TON of the metal catalyst is 500,000 or more. As another example, it is preferable that the yield is more than 55% and the TON of the metal catalyst is 300,000 or more.

### Method for Producing Formic Acid

The method for producing formic acid according to the present embodiment includes a step of producing the formate by the above-described method for producing the formate, and a step of protonating at least a part of the formate to generate formic acid. In the description herein, the step of protonating at least a part of formate to generate formic acid may be referred to as second step. The method for producing formic acid according to the present embodiment includes, for example, the above-described first step and the second step.

In the first step, since the generated formate is eluted into the aqueous phase, the aqueous solution of formate is obtained by fractionating the aqueous phase. Preferably, the aqueous phase in the first step is separated, and the obtained aqueous solution is processed by using, for example, an electrodialyzer to generate formic acid in the second step. The aqueous phase to be separated is the aqueous phase obtained after the first step has ended.

In the second step, the aqueous solution of the formate obtained in the first step may be used as it is, or may be concentrated or diluted as necessary to adjust the concentration of formate in the aqueous solution, and used. Examples of the method for diluting the aqueous solution of formate include a method in which the aqueous solution of formate is diluted by adding pure water. Examples of the method for concentrating the aqueous solution of formate include a method in which water is distilled off from the aqueous solution, and a method in which the aqueous solution is concentrated by using a separation membrane unit having a reverse osmosis membrane. In a case where the process is performed by using an electrodialyzer, loss of formate may occur due to a phenomenon of concentration diffusion in the aqueous solution of formate which has a high concentration. From the viewpoint of reducing the loss, it is preferable that the aqueous phase in the first step is separated, and the concentration of the formate is adjusted by dilution, and thereafter, the obtained aqueous solution is used in the second step. In a case where, in the first step, the aqueous solution of formate which has a high concentration is produced, and the concentration of the aqueous solution is adjusted by dilution, and then, the obtained aqueous solution is used in the second step, formic acid can be produced at a higher yield with more excellent productivity.

A degree of adjustment (preferably, dilution) of the concentration of the aqueous solution of formate which is obtained in the first step is not particularly limited. The concentration of formate in the aqueous solution after the adjustment of the concentration is preferably the concentration suitable for electrodialysis, and is preferably 2.5 mol/L or more, more preferably 3 mol/L or more and 4.75 mol/L or more, and even more preferably 5 mol/L or more. In a case where the process is performed by using an electrodialyzer, the concentration of formate is preferably 20 mol/L or less, more preferably 15 mol/L or less, and even more preferably 10 mol/L or less from the viewpoint of reducing loss of formate due to a phenomenon of concentration diffusion.

Pure water can be used for dilution. Water generated in the second step may be used for dilution. Reusing water generated in the second step for dilution is preferable since, for example, cost for waste water treatment and a load on the environment can be advantageously reduced.

In the method for producing formic acid according to the present embodiment, acid is added to the aqueous solution of formate which is obtained in the first step, and decarbonation treatment is performed, and thereafter, the resultant aqueous solution may be used in the second step. That is, the aqueous phase in the first step is separated, acid is added, and the decarbonation treatment is performed, and thereafter, the resultant aqueous solution may be used in the second step. The aqueous solution of formate which is obtained in the first step may contain unreacted carbonate or hydrogencarbonate generated by a side reaction, and, if a solution containing carbonate or hydrogencarbonate is subjected to electrodialysis, carbon dioxide may be generated and dialysis efficiency may be reduced. Therefore, acid is added to the aqueous solution of formate which is obtained in the first step, and decarbonation treatment is performed, and thereafter, electrodialysis is performed, whereby formic acid can be produced at a higher yield with more excellent productivity.

Examples of the acid used for the decarbonation treatment include formic acid, citric acid, acetic acid, malic acid, lactic acid, succinic acid, tartaric acid, butyric acid, fumaric acid, propionic acid, hydrochloric acid, nitric acid, and sulfuric acid. Formic acid is preferably used.

An amount of the acid to be used is preferably 50% or more and more preferably 80% or more with respect to an amount of carbonic acid existing in the solution from the viewpoint of reducing an amount of carbonic acid generated during electrodialysis treatment. Furthermore, in a case where a pH of the solution of formate is set to be approximately neutral during electrodialysis treatment, deterioration of an electrodialyzer can be inhibited. Therefore, an amount of the acid to be used is preferably 150% or less and more preferably 120% or less with respect to an amount of carbonic acid existing in the solution.

In the present embodiment, as a proportion at which formate is protonated in the second step, preferably 10% or more, more preferably 20% or more, and even more preferably 30% or more of formate is protonated with respect to an initial molar amount of formate in the aqueous solution of formate, from the viewpoint of enhancing purity of the aqueous solution of formic acid which is to be collected.

Examples of the electrodialyzer used in the second step include a two-chamber type electrodialyzer in which a bipolar membrane, and an anion exchange membrane or a cation exchange membrane are used, and a three-chamber-type electrodialyzer in which a bipolar membrane, an anion exchange membrane, and a cation exchange membrane are used.

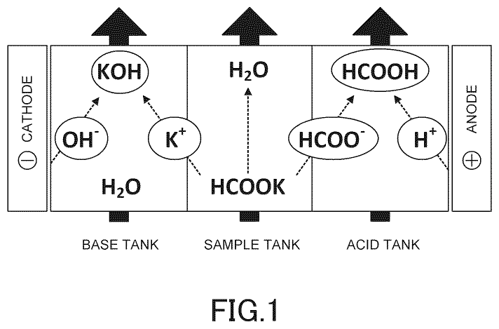

FIG. 1 is a schematic diagram illustrating one example of a three-chamber-type electrodialyzer. The electrodialyzer illustrated in FIG. 1 includes a plurality of bipolar membranes, a plurality of anion exchange membranes, and a plurality of cation exchange membranes. The bipolar membranes, the anion exchange membranes, and the cation exchange membranes are disposed between an anode and a cathode, so that a base tank, a sample tank (salt tank), and an acid tank are formed. The aqueous solution of formate is circulated and supplied to the sample tank while electricity is caused to pass through the electrodialyzer, so that formate can be converted to formic acid, formic acid can be collected from the acid tank, water can be collected from the sample tank, and hydroxide can be collected from the base tank.

The two-chamber-type electrodialyzer includes, for example, a plurality of bipolar membranes and a plurality of cation exchange membranes. The bipolar membranes and the cation exchange membranes alternate between an anode and a cathode, so that a salt chamber is formed between each bipolar membrane and the cation exchange membrane disposed on the cathode side of the bipolar membrane, and a base tank is formed between each bipolar membrane and the cation exchange membrane disposed on the anode side of the bipolar membrane. The aqueous solution of formate is circulated and supplied to the salt chamber while electricity is caused to pass through the electrodialyzer, so that hydroxide is generated in the base tank, and formate that is circulated and supplied to the salt chamber is converted to formic acid.

In the second step, by using the electrodialyzer, formate can be protonated by a simple method to obtain a solution of formic acid.

### System for Producing Formic Acid

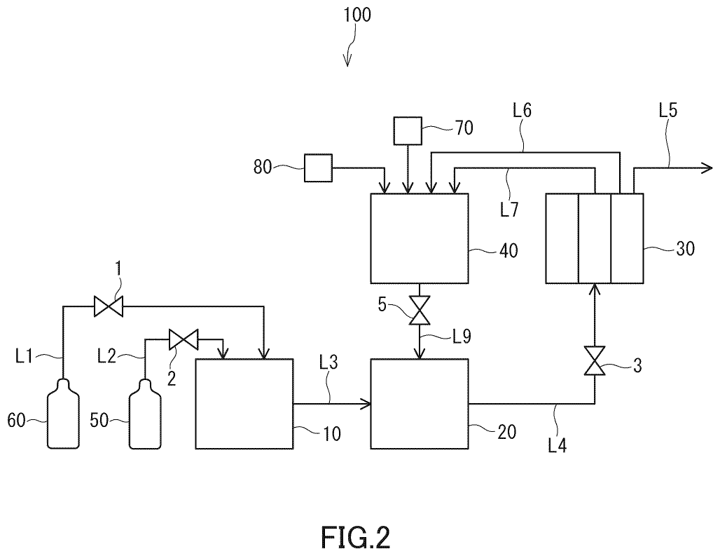

As shown in FIG. 2, a production system 100 for producing formic acid according to the present embodiment includes, for example, a production device 10 for producing formate, and an electrodialyzer 30. The production system 100 may further include a diluting device 20 and a storage portion 40 for storing dilution water, and may further include a carbon dioxide cylinder 60 for introducing carbon dioxide into the production device 10, and a hydrogen cylinder 50 for introducing hydrogen into the production device 10. The concentration and the pressure of each of carbon dioxide and hydrogen can be adjusted by a valve 1 and a valve 2 disposed in piping L1 and piping L2, respectively.

Formate produced by the production device 10 is supplied to the electrodialyzer 30 as the aqueous solution of formate by separating the aqueous phase. At this time, as shown in FIG. 2, the concentration of formate in the aqueous solution may be adjusted by sending the aqueous solution of formate to the diluting device 20 beforehand through a flow path L3, and diluting the aqueous solution of formate by the diluting device 20.

In the aqueous solution in which the concentration of formate has been adjusted by the diluting device 20, at least a part of formate is protonated by the electrodialyzer 30. Thus, formic acid and water are generated from formate. The generated formic acid can be taken out through a flow path L5. The generated water may be sent to the storage portion 40 through a flow path L7.

A part of formic acid generated by the electrodialyzer 30 may be sent to the storage portion 40 through a flow path L6. The storage portion 40 may further include a water supply portion 70 and a formic acid supply portion 80. By supplying the aqueous solution of formic acid which has been prepared by the storage portion 40 to the diluting device 20 through a flow path L9, the aqueous solution of formate may be subjected to decarbonation treatment. Each of flow paths of the production system 100 may include a valve for adjusting pressure and a supply amount.

The production system 100 of the present embodiment can produce formic acid at a high yield with excellent productivity.

### EXAMPLES

The present invention will be more specifically described below by way of examples and comparative examples. However, the present invention is not limited to the examples.

### Synthesis of Metal Catalyst

### Synthesis Example 1

### Synthesis of Ru Catalyst 1

A Ru catalyst 1 was synthesized by the following operation. Firstly, 40 mg (0.1 mmol) of the following ligand A was added to a THF (tetrahydrofuran) (5 ml) suspension of 95.3 mg (0.1 mmol) of [RuHCl(PPh₃)₃(CO)] in an inert atmosphere, and the mixture was stirred and heated at 65° C. for three hours, to cause a reaction. Thereafter, the resultant product was cooled to room temperature (25° C.). The obtained yellow solution was filtered, and the filtrate was evaporated to dryness under a vacuum. The obtained yellow residual oil was dissolved in a small amount of THF (1 mL), and hexane (10 mL) was slowly added to precipitate a yellow solid product, and the solid product was filtered. The filtrate was dried under a vacuum, and the following Ru catalyst 1 (55 mg, yield of 97%) was obtained as yellow crystals. In the following Ru catalyst 1 and ligand A, tBu represents a tertiary butyl group.

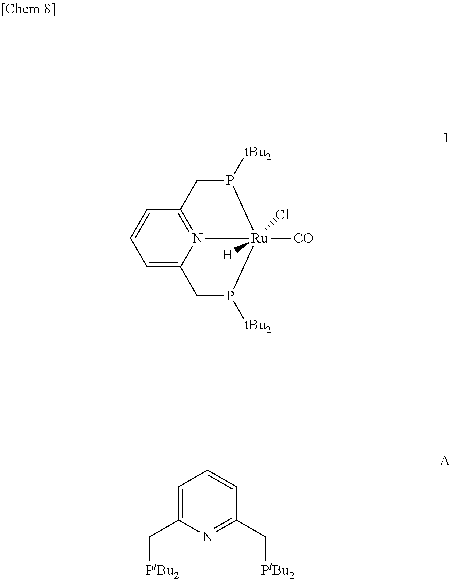

³¹P{¹H}(C₆D₆): 90.8 (s), 1H (C₆D₆): −14.54 (t, 1H, J=20.0 Hz), 1.11 (t, 18H, J=8.0 Hz), 1.51 (t, 18H, J=8.0 Hz), 2.88 (dt, 2H, J=16.0 Hz, J=4.0 Hz), 3.76 (dt, 2H, J=16.0 Hz, J=4.0 Hz), 6.45 (d, 2H, J=8.0 Hz), 6.79 (t, 1H, J=8.0 Hz). ¹³C{¹H}NMR(C₆D₆): 29.8(s), 30.7(s), 35.2 (t, J=9.5 Hz), 37.7 (t, J=6.0 Hz), 37.9 (t, J=6.5 Hz), 119.5 (t, J=4.5 Hz), 136.4(s), 163.4 (t, J=5.0 Hz), 209.8(s).

For the Ru catalyst 1, the catalyst turnover number e was calculated as an index of catalytic activity by the above-described testing method. As a result, the Ru catalyst 1 had the catalyst turnover number e of 27500.

### Calculation of TON and Yield

In the following examples and comparative examples, the TON of the metal catalyst and the yield of formate (potassium formate) were calculated by the following methods. Firstly, 100 μL of the aqueous phase (aqueous solution) obtained after the reaction was taken out, and was dissolved in 500 μL of Deuterium oxide, and 300 μL of dimethyl sulfoxide was further added as an internal standard, to produce a measurement sample. For the measurement sample, ¹H NMR measurement was performed, and a substance amount X of formate included in the aqueous solution was quantified by the same method as described above for the catalyst turnover number e. The TON of the metal catalyst was calculated according to the following equation based on the substance amount X (mol) of the formate and a substance amount Y (mol) of the metal catalyst used for the reaction.

$$TON of metal catalyst = X / Y$$

Furthermore, the yield (%) of formate was calculated according to the following equation based on the substance amount X (mol) of the formate and a substance amount Z (mol) of the compound C used for the reaction.

$$Yield of formate = 100 \times X / Z$$

### Example 1

In a glove box in inert gas, 1 ml of water was weighed into a glass vial having a stirring rod, and 5 mmol of potassium hydrogencarbonate was added. Subsequently, 1 mL of toluene, 0.005 μmol of the Ru catalyst 1, and 54 μmol of methyltrioctylammonium chloride were mixed, and the obtained mixed solution was added into the vial. The vial was set in an autoclave, and the autoclave was sealed, and then taken out from the glove box.

Subsequently, the mixture in the vial was heated to 90° C. while being stirred. When the temperature of the mixture reached 90° C., the autoclave was pressurized to 4.5 MPa with hydrogen, and the mixture was further stirred for 60 hours. Thus, the reaction between hydrogen and potassium hydrogencarbonate progressed. The reaction solution was cooled by using an ice bath, and then, the pressure in the autoclave was carefully released. By removing the upper layer (organic phase) of the reaction solution, the aqueous phase (aqueous solution) as the lower layer was obtained. By using the aqueous solution, the TON of the metal catalyst and the yield of formate were calculated by the above-described method.

### Example 2

In a glove box in inert gas, 1 ml of water was weighed into a glass vial having a stirring rod, and 5 mmol of potassium hydrogencarbonate was added. Subsequently, 1 mL of toluene, 0.004 μmol of the Ru catalyst 1, 0.0264 μmol of the ligand A, and 54 μmol of methyltrioctylammonium chloride were mixed, and the obtained mixed solution was added into the vial. The vial was set in an autoclave, and the autoclave was sealed, and then taken out from the glove box.

Subsequently, the mixture in the vial was heated to 90° C. while being stirred. When the temperature of the mixture reached 90° C., the autoclave was pressurized to 5.0 MPa with hydrogen, and the mixture was further stirred for 46 hours. The reaction solution was cooled by using an ice bath, and then, the pressure in the autoclave was carefully released. By removing the upper layer (organic phase) of the reaction solution, the aqueous phase (aqueous solution) as the lower layer was obtained. By using the aqueous solution, the TON of the metal catalyst and the yield of formate were calculated by the above-described method.

### Example 3

In a glove box in inert gas, 1 ml of water was weighed into a glass vial having a stirring rod, and 5 mmol of potassium hydrogencarbonate was added. Subsequently, 1 mL of toluene, 0.004 μmol of the Ru catalyst 1, 0.0352 μmol of the ligand A, and 54 μmol of methyltrioctylammonium chloride were mixed, and the obtained mixed solution was added into the vial. The vial was set in an autoclave, and the autoclave was sealed, and then taken out from the glove box.

Subsequently, the mixture in the vial was heated to 90° C. while being stirred. When the temperature of the mixture reached 90° C., the autoclave was pressurized to 5.0 MPa with hydrogen, and the mixture was further stirred for 46 hours. The reaction solution was cooled by using an ice bath, and then, the pressure in the autoclave was carefully released. By removing the upper layer (organic phase) of the reaction solution, the aqueous phase (aqueous solution) as the lower layer was obtained. By using the aqueous solution, the TON of the metal catalyst and the yield of formate were calculated by the above-described method.

### Example 4

In a glove box in inert gas, 1 ml of water was weighed into a glass vial having a stirring rod, and 5 mmol of potassium hydrogencarbonate was added. Subsequently, 1 mL of toluene, 0.006 μmol of the Ru catalyst 1, and 54 μmol of methyltrioctylammonium chloride were mixed, and the obtained mixed solution was added into the vial. The vial was set in an autoclave, and the autoclave was sealed, and then taken out from the glove box.

Subsequently, the mixture in the vial was heated to 90° C. while being stirred. When the temperature of the mixture reached 90° C., the autoclave was pressurized to 4.5 MPa with hydrogen, and the mixture was further stirred for 60 hours. The reaction solution was cooled by using an ice bath, and then, the pressure in the autoclave was carefully released. By removing the upper layer (organic phase) of the reaction solution, the aqueous phase (aqueous solution) as the lower layer was obtained. By using the aqueous solution, the TON of the metal catalyst and the yield of formate were calculated by the above-described method.

### Example 5

In a glove box in inert gas, 1 ml of water was weighed into a glass vial having a stirring rod, and 5 mmol of potassium hydrogencarbonate was added. Subsequently, 1 mL of toluene, 0.006 μmol of the Ru catalyst 1, 0.03 μmol of the ligand A, and 54 μmol of methyltrioctylammonium chloride were mixed, and the obtained mixed solution was added into the vial. The vial was set in an autoclave, and the autoclave was sealed, and then taken out from the glove box.

Subsequently, the mixture in the vial was heated to 90° C. while being stirred. When the temperature of the mixture reached 90° C., the autoclave was pressurized to 4.5 MPa with hydrogen, and the mixture was further stirred for 48 hours. The reaction solution was cooled by using an ice bath, and then, the pressure in the autoclave was carefully released. By removing the upper layer (organic phase) of the reaction solution, the aqueous phase (aqueous solution) as the lower layer was obtained. By using the aqueous solution, the TON of the metal catalyst and the yield of formate were calculated by the above-described method.

### Comparative Example 1

In a glove box in inert gas, 1 ml of water was weighed into a glass vial having a stirring rod, and 5 mmol of potassium hydrogencarbonate was added. Subsequently, 1 mL of toluene, 0.012 μmol of the Ru catalyst 1, and 54 μmol of methyltrioctylammonium chloride were mixed, and the obtained mixed solution was added into the vial. The vial was set in an autoclave, and the autoclave was sealed, and then taken out from the glove box.

Subsequently, the mixture in the vial was heated to 90° C. while being stirred. When the temperature of the mixture reached 90° C., the autoclave was pressurized to 4.0 MPa with hydrogen, and the mixture was further stirred for 18 hours. The reaction solution was cooled by using an ice bath, and then, the pressure in the autoclave was carefully released. By removing the upper layer (organic phase) of the reaction solution, the aqueous phase (aqueous solution) as the lower layer was obtained. By using the aqueous solution, the TON of the metal catalyst and the yield of formate were calculated by the above-described method.

### Comparative Example 2

In a glove box in inert gas, 1 ml of water was weighed into a glass vial having a stirring rod, and 5 mmol of potassium hydrogencarbonate was added. Subsequently, 1 mL of toluene, 0.029 μmol of the Ru catalyst 1, and 54 μmol of methyltrioctylammonium chloride were mixed, and the obtained mixed solution was added into the vial. The vial was set in an autoclave, and the autoclave was sealed, and then taken out from the glove box.

Subsequently, the mixture in the vial was heated to 90° C. while being stirred. When the temperature of the mixture reached 90° C., the autoclave was pressurized to 4.0 MPa with hydrogen, and the mixture was further stirred for 18 hours. The reaction solution was cooled by using an ice bath, and then, the pressure in the autoclave was carefully released. By removing the upper layer (organic phase) of the reaction solution, the aqueous phase (aqueous solution) as the lower layer was obtained. By using the aqueous solution, the TON of the metal catalyst and the yield of formate were calculated by the above-described method.

### Comparative Example 3

In a glove box in inert gas, 1 ml of water was weighed into a glass vial having a stirring rod, and 5 mmol of potassium hydrogencarbonate was added. Subsequently, 1 mL of toluene, 0.059 μmol of the Ru catalyst 1, and 54 μmol of methyltrioctylammonium chloride were mixed, and the obtained mixed solution was added into the vial. The vial was set in an autoclave, and the autoclave was sealed, and then taken out from the glove box.

Subsequently, the mixture in the vial was heated to 90° C. while being stirred. When the temperature of the mixture reached 90° C., the autoclave was pressurized to 4.0 MPa with hydrogen, and the mixture was further stirred for 18 hours. The reaction solution was cooled by using an ice bath, and then, the pressure in the autoclave was carefully released. By removing the upper layer (organic phase) of the reaction solution, the aqueous phase (aqueous solution) as the lower layer was obtained. By using the aqueous solution, the TON of the metal catalyst and the yield of formate were calculated by the above-described method.

### Comparative Example 4

In a glove box in inert gas, 1 ml of water was weighed into a glass vial having a stirring rod, and 5 mmol of potassium hydrogencarbonate was added. Subsequently, 1 mL of toluene, 0.12 μmol of the Ru catalyst 1, and 54 μmol of methyltrioctylammonium chloride were mixed, and the obtained mixed solution was added into the vial. The vial was set in an autoclave, and the autoclave was sealed, and then taken out from the glove box.

Subsequently, the mixture in the vial was heated to 90° C. while being stirred. When the temperature of the mixture reached 90° C., the autoclave was pressurized to 4.0 MPa with hydrogen, and the mixture was further stirred for 18 hours. The reaction solution was cooled by using an ice bath, and then, the pressure in the autoclave was carefully released. By removing the upper layer (organic phase) of the reaction solution, the aqueous phase (aqueous solution) as the lower layer was obtained. By using the aqueous solution, the TON of the metal catalyst and the yield of formate were calculated by the above-described method.

TABLE 1

| | | Yield | | | | | | | | |
|---|---|---|---|---|---|---|---|---|---|---|
| | TON | (%) | y | x₁ | x₂ | x₃ | x₄ | x₅ | x₆ | x₇ |
| Example 1 | 557569 | 53 | 5.7 | 200 | 0 | 0.001 | 0 | 20 | 1.82 × 10⁻⁷ | 1650000 |
| Example 2 | 546814 | 43 | 5.7 | 250 | 6.6 | 8.00 × 10⁻⁴ | 0.0264 | 18 | 1.45 × 10⁻⁷ | 1265000 |
| Example 3 | 794999 | 62 | 5.6 | 250 | 8.8 | 8.00 × 10⁻⁴ | 0.0352 | 18 | 1.45 × 10⁻⁷ | 1265000 |
| Example 4 | 502978 | 57 | 5.6 | 166.7 | 0 | 0.0012 | 0 | 20 | 2.18 × 10⁻⁷ | 1650000 |
| Example 5 | 660998 | 80 | 5.5 | 166.7 | 5 | 0.0012 | 0.03 | 20 | 2.18 × 10⁻⁷ | 1320000 |
| Comparative | 296756 | 70 | 5.2 | 83.3 | 0 | 0.0024 | 0 | 23 | 4.36 × 10⁻⁷ | 495000 |
| example 1 | | | | | | | | | | |
| Comparative | 109224 | 64 | 4.94 | 34.5 | 0 | 0.0058 | 0 | 23 | 1.05 × 10⁻⁶ | 495000 |
| example 2 | | | | | | | | | | |
| Comparative | 56996 | 67 | 4.77 | 16.9 | 0 | 0.0118 | 0 | 23 | 2.15 × 10⁻⁶ | 495000 |
| example 3 | | | | | | | | | | |
| Comparative | 27728 | 67 | 4.51 | 8.33 | 0 | 0.024 | 0 | 23 | 4.36 × 10⁻⁶ | 495000 |
| example 4 | | | | | | | | | | |

As indicated in Table 1, in the examples in which the reaction between hydrogen and the compound C was performed under a condition that the value of y calculated by the above-described Equation (I) was more than 5.2, the TON of the metal catalyst was improved as compared with the comparative examples. Furthermore, under the condition in each of the examples, the yield of formate was 40% or more, and the value was sufficient for practical use.

### Protonation of Formate

In the following Reference examples 1 and 2, formate was protonated by using an electrodialyzer (ACILYZER EX3B manufactured by ASTOM Corporation).

### Reference Example 1

In a base tank of the electrodialyzer, 165 g of potassium hydroxide which was dissolved in 500 ml of water was put. In a salt tank, 500 mL of a 5 mol/L potassium formate aqueous solution was put. In an acid tank, 500 mL of a 4.35 mol/L formic acid aqueous solution was put. Electrodialysis was performed at a voltage of 28 V for 80 minutes. After the dialysis ended, 100 μL of the solution (acid liquid) in the acid tank was taken and dissolved in 500 μL of Deuterium oxide, and 300 μL of dimethyl sulfoxide was additionally added as an internal standard, and ¹H NMR measurement was performed, to quantify formic acid in the acid liquid having been subjected to the dialysis.

### Reference Example 2

In a base tank of the electrodialyzer, 165 g of potassium hydroxide which was dissolved in 500 ml of water was put. In a salt tank, 500 mL of an aqueous solution containing 4.75 mol/L of potassium formate and 0.25 mol/L of potassium hydrogencarbonate was put. In an acid tank, 500 mL of a 4.81 mol/L formic acid aqueous solution was put. Electrodialysis was performed at a voltage of 28 V for 80 minutes. After the dialysis ended, 100 μL of the solution (acid liquid) in the acid tank was taken and dissolved in 500 μL of Deuterium oxide, and 300 μL of dimethyl sulfoxide was additionally added as an internal standard, and ¹H NMR measurement was performed, to quantify formic acid in the acid liquid having been subjected to the dialysis.

Indication in Table 2 is for Reference examples 1 and 2. In Table 2, an initial formate proportion represents a percentage of a substance amount X2 of formate relative to the total of the substance amount (substance amount X2) of formate and a substance amount of hydrogencarbonate in the salt tank prior to the electrodialysis. An initial formate concentration represents a molar concentration of formate in the salt tank prior to the electrodialysis. An initial hydrogencarbonate concentration represents a molar concentration of hydrogencarbonate in the salt tank prior to the electrodialysis. An initial formic acid concentration represents a molar concentration of formic acid in the acid tank prior to the electrodialysis. A final formic acid concentration represents a molar concentration of formic acid in the acid tank after the end of the electrodialysis. A yield of formic acid represents a percentage of a substance amount (mol) of formic acid obtained through the electrodialysis, relative to the substance amount (substance amount X2) of formate used for the electrodialysis.

TABLE 2

| | | Reference | Reference |
|---|---|---|---|
| | | example 1 | example 2 |
| Prior to | Initial formate proportion (%) | 100 | 95 |
| dialysis | Initial formate concentration (mol/L) | 5 | 4.75 |
| | Initial hydrogencarbonate concentration | 0 | 0.25 |
| | (mol/L) | | |
| | Initial formic acid concentration (mol/L) | 4.35 | 4.81 |
| After | Final formic acid concentration (mol/L) | 6.28 | 6.68 |
| dialysis | Yield of formic acid (%) | 50.3 | 51.8 |

As indicated in Table 2, formic acid was able to be obtained from formate through the electrodialysis. In Reference example 2 in which hydrogencarbonate was contained in the salt tank, formic acid was able to be obtained at a yield equivalent to that in Reference example 1 in which no hydrogencarbonate was contained in the salt tank.

### INDUSTRIAL APPLICABILITY

In the method for producing the formate according to the present embodiment, for example, the formate as a precursor of formic acid can be efficiently produced.

1. A method for producing a formate through reaction between hydrogen, and a compound C including at least one selected from the group consisting of carbon dioxide, hydrogencarbonate, and carbonate in the presence of a solvent by using a metal catalyst, wherein
the solvent includes an organic solvent and an aqueous solvent,
a ligand is added to the solvent as necessary,
the reaction between hydrogen and the compound C is performed in a two-phase system in which the organic solvent and the aqueous solvent are separate under a condition that a value of y calculated by Equation (I) indicated below is more than 5.2,
$$y = 0.00398 {x}_{1} + ( - 0.0598 ) {x}_{2} + ( - 15.1 ) {x}_{3} + 6.66 {x}_{4} + ( - 0.00340 ) {x}_{5} + ( - 157 \; 99 ) {x}_{6} + 3.43 \times 1 {0}^{- 8} {x}_{7} + 4 \text{.97} , \qquad ( I )$$
in Equation (I), x₁ represents a reciprocal 1/a of a ratio a (mmol/L) of a substance amount of the metal catalyst to a volume of the organic solvent,
x₂ represents a ratio b of a substance amount of the ligand to the substance amount of the metal catalyst,
x₃ represents a value a/c obtained by dividing the ratio a (mmol/L) by a ratio c (mol/L) of a substance amount of the compound C that can be used for the reaction, to a volume of the aqueous solvent,
x₄ represents a value a×b obtained by multiplying the ratio a by the ratio b,
x₅ represents a ratio d of a reaction temperature (° C.) to a pressure (MPa) of the hydrogen,
x₆ represents a value a/e obtained by dividing the ratio a (mmol/L) by a catalyst turnover number e of the metal catalyst which is calculated in a test described below, and
x₇ represents a value e×f obtained by multiplying the catalyst turnover number e by a reaction time f (h),
Test: 1 mL of water and 5 mmol of potassium hydrogencarbonate are added into a vial having a stirring rod in an inert gas atmosphere, and 1 mL of toluene, 0.12 μmol of the metal catalyst, and 54 μmol of methyltrioctylammonium chloride are further added, the vial is set in an autoclave, the autoclave is sealed, a mixture in the vial is heated to 90° C. while being stirred, the autoclave is pressurized to 4.5 MPa with hydrogen when the temperature of the mixture has reached 90° C., the mixture is further stirred for 18 hours, the mixture is cooled, the vial is taken out from the autoclave, a substance amount of potassium formate included in the mixture is quantified, and the catalyst turnover number e of the metal catalyst is calculated from the obtained value.

2. The method for producing a formate according to claim 1, wherein the value of y is 5.7 or more.

3. The method for producing a formate according to claim 1, wherein a value of the x₁ is 100 or more.

4. The method for producing a formate according to claim 1, wherein the value of x₃ is 0.002 or less.

5. The method for producing a formate according to claim 1, wherein the value of x₄ is 0.028 or more.

6. The method for producing a formate according to claim 1, wherein a value of the x₅ is 20 or less.

7. The method for producing a formate according to claim 1, wherein the value of x₆ is 4.00×10⁻⁷ or less.

8. The method for producing a formate according to claim 1, wherein the value of x₇ is 500,000 or more.

9. The method for producing a formate according to claim 1, wherein the metal catalyst includes at least one selected from the group consisting of ruthenium and iridium.

10. The method for producing a formate according to claim 1, wherein the metal catalyst includes at least one selected from the group consisting of a ruthenium complex represented by General formula (1) indicated below, a tautomer of the ruthenium complex, a stereoisomer of the ruthenium complex, and salt compounds thereof, 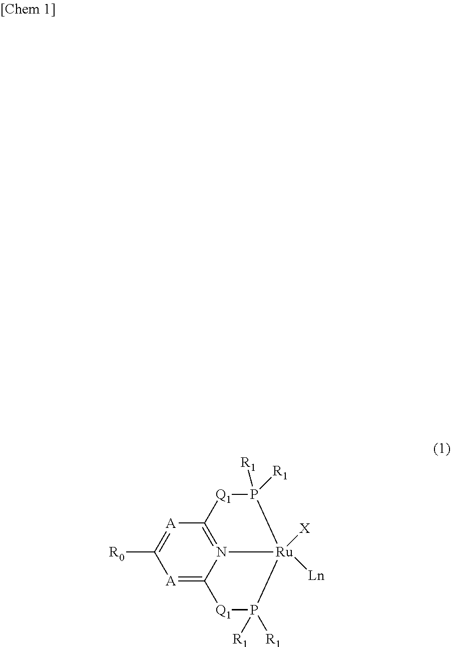
(in General formula (1), R₀ represents a hydrogen atom or an alkyl group,
each Q₁ independently represents CH₂, NH, or O,
each R₁ independently represents an alkyl group or an aryl group (in a case where Q₁ represents NH or O, at least one R₁ represents an aryl group),
each A independently represents CH, CR₅, or N,
R₅ represents an alkyl group, an aryl group, an aralkyl group, an amino group, a hydroxy group, or an alkoxy group,
X represents a halogen atom,
n represents 0 to 3, and
in a case where the number of Ls is plural, each L independently represents a neutral or anionic ligand).

11. The method for producing a formate according to claim 1, wherein the organic solvent includes toluene.

12. The method for producing a formate according to claim 1, wherein the compound C includes potassium hydrogencarbonate.

13. The method for producing a formate according to claim 1, wherein a quaternary ammonium salt is further used as a phase transfer catalyst in the reaction.

14. A method for producing formic acid comprising:
producing the formate by the method for producing a formate according to claim 1; and
protonating at least a part of the formate to generate formic acid.
